(19) United States
(12) Patent Application Publication
(10) Pub. No.: US 2010/0240065 A1
(43) Pub. Date: Sep. 23, 2010

(54) Prolyl Hydroxylase Compositions and Methods of Use Thereof

(75) Inventors: John Alan Broadwater, Ridgefield, CT (US); Richard D. Schneiderman, Ridgefield, CT (US); Kelli-Ann Debra Monaco, Ridgefield, CT (US)

Correspondence Address: Michael P. Morris;Boehringer Ingelheim USA Corporation, 900 Ridgebury Road, Ridgefield, CT, 06877-0368, US

(73) Assignee: Boehringer Ingelheim International GmbH, Ingelheim (DE)

(21) Appl. No.: 12/406,679

(22) Filed: Mar. 18, 2009

(51) Int. Cl. G01N 33/53; C07H 21/00; C12N 15/74; C12N 1/00; C07K 14/00; C12P 21/06

(52) U.S. Cl. 435/7.1; 536/23.1; 435/320.1; 435/243; 530/350; 435/69.1

(57) ABSTRACT

The invention provides methods and compositions for making and using novel HIF-specific prolyl hydroxylase (HPH) enzymes from rhesus monkeys.

### RELATED APPLICATIONS

[Not Applicable]

### FIELD OF THE INVENTION

The present invention relates to novel genes encoding for prolyl hydroxylases and methods and compositions for making and using the same.

### BACKGROUND OF THE INVENTION

Hypoxia, physiologically the condition in which an animal's oxygen demand exceeds the supply, is known to have detrimental effects in various conditions. For example, regions of hypoxia are common in breast carcinoma where the rate of nutrient and oxygen consumption is insufficient to meet the metabolic demands of neoplastic cells. Other systemic, local, and intracellular homeostatic responses elicited by hypoxia include angiogenesis, erythropoiesis, neovascularization in ischemic myocardium, and glycolysis in cells cultured at reduced O₂ tension, vasomotor control, inflammation, tissue matrix metabolism, cell survival decisions, and tissue ischemia resulting from chronic hypoxia occurring from, for example, stroke, deep vein thrombosis, pulmonary embolus, and renal failure. lschemic tissue is also found in tumors.

The adaptive responses to hypoxia either increase O₂ delivery or activate alternate metabolic pathways that do not require O₂. Hypoxia-inducible gene products that participate in these responses include erythropoietin (EPO), vascular endothelial growth factor (VEGF), and glycolytic enzymes. Hypoxia-inducible factor (HIF) is a transcription factor that plays a central role in cellular adaptation to hypoxia. HIF-1 is a heterodimer consisting of two subunits, HIF-1 and HIF-1β. The HIF-1α subunit is unique to HIF-1, whereas HIF-1 β (also known as aryl hydrocarbon receptor nuclear translocator, ARNT) can dimerize with other proteins.

HIF-1 β is constitutively expressed whereas HIF-1 α-subunits are tightly regulated. Under normal oxygen conditions HIF 1α also is constitutively expressed but it is tightly regulated and is rapidly degraded in a proteosome-mediated pathway via a protein-ubiquitin ligase complex containing the product of the von Hippel Lindau tumor suppressor protein. pVHL recognizes an oxygen degradation domain of HIF 1α only under normal oxygen conditions.

Under hypoxic conditions, HIF-1α is not hydroxylated because the hydroxylase, which requires dioxygen for activity, is inactive and thus HIF-1α is not recognized by pVHL and accumulates in the cell. HIF-1α then translocates to the nucleus and dimerizes with the constitutively present HIF-1β subunit (Semenza, Genes & Development, 1985, 14: 1983 1991). The dimer then binds to the hypoxia responsive element (HRE) in target genes, resulting in their transactivation of genes such as erythropoietin, VEGF (Forsythe et al., Mol. Cell. Biol., 1996, 16: 4604 4613), platelet-derived growth factor-β (PDGF-β), glucose transporter (GLUT1) and nitrous oxide synthetase (Neckers, J. Natl. Cancer Ins., 1999, 91: 106 107). Certain hormones and growth factors also lead to increased levels of HIF-1α as well as mutations in certain oncogenes and tumor-suppressor genes, VHL for example, result in an increase in HIF-1α level (Ivan and Kaelin, Current Opinion in Genetics & Development, 2001, 11: 27 34). It will be interesting to determine whether hydroxylation or alternative mechanisms are involved in this hypoxia-independent HIF activation.

VHL recognizes the oxygen degradation domain via a conserved proline residue that is hydroxylated exclusively under normal oxygen conditions. Under normoxic conditions there is a prolyl-4-hydroxylase activity capable of modifying a proline-containing peptide derived from the oxygen degradation domain of HIF-1a. However, this activity is greatly diminished under hypoxic conditions. As is the case for known prolyl-4-hydroxylases, this activity was enhanced by supplementation with Fe²⁺, ascorbate and 2-oxoglutarate.

An evolutionarily and structurally conserved family of HIF prolyl hydroxylase (HPH) enzymes has been described (U.S. Pat. No. 6,566,088). However, there remains a need to identify additional such enzymes that can be readily used to identify agents that can be used in the treatment of various hypoxia-related disorders.

### BRIEF SUMMARY OF THE INVENTION

The invention provides methods and compositions for assaying hypoxia-inducible factor (HIF) prolyl hydroxylation and novel HIF-specific prolyl hydroxylase (HPH) enzymes for use therein. The assays may be cell-based or in vitro assays employing compositions comprising the isolated proteins. The assays comprise incubating a mixture comprising an isolated or recombinantly expressed HIF-specific prolyl hydroxylase (HPH), and a substrate of the hydroxylase, under conditions whereby the prolyl hydroxylase hydroxylates the substrate, and detecting a resultant prolyl hydroxylation of the substrate. The mixture may also comprise a candidate agent which modulates the resultant prolyl hydroxylation. In particular embodiments, the hydroxylase is selected from the group consisting of human HPH1, HPH2 and HPH3, and/or the substrate comprises LAPY, wherein P is hydroxylated by the hydroxylase of the present invention.

More particularly, the invention relates to an isolated nucleic acid sequence of SEQ ID NO: 1, SEQ ID NO:3 or SEQ ID NO:5. Alternative embodiments describe an isolated nucleic acid sequence that encodes a protein of SEQ ID NO:2, SEQ ID NO:4 or SEQ ID NO:6. Also contemplated herein are expression constructs comprising the nucleic acids of the invention operably linked to a promoter; expression vectors comprising such nucleic acids; and host cells transformed or transfected with such expression vectors under conditions that allow expression of the nucleic acid in the host cell; as well as methods of use thereof in the recombinant production of proteins encoded by the nucleic acids contained in the expression vectors.

Also contemplated are isolated and purified protein compositions in which the protein comprises a sequence of SEQ ID NO:2, SEQ ID NO:4 or SEQ ID NO:6. Preferably, in such compositions, the isolated protein is conjugated to a detectable label.

An exemplary production method of the invention is a method of producing a HIF prolyl hydroxylase protein comprising transforming a host cell with an expression vector under condition to allow expression of the nucleic acid sequence contained in the expression vector by the host cell; culturing the host cell to produce a protein product of the expressed nucleic acid sequence and isolating the protein product.

Also contemplated are methods of increasing expression of a hypoxia inducible gene in a cell comprising contacting a cell that comprises a hypoxia inducible gene with an expression vector described herein under conditions that allow expression of the nucleic acid sequence contained in the vector thereby providing for increased expression of a hypoxia inducible gene in the cell.

The present invention also provides novel methods of assaying for hypoxia-inducible factor (HIF) prolyl hydroxylation, comprising the steps of: a) incubating a mixture comprising an isolated HIF-specific prolyl hydroxylase (HPH) protein or recombinantly expressed HPH selected from the group consisting of a protein of SEQ ID NO:2, SEQ ID NO:4 and SEQ ID NO: 6, and a peptide substrate of HPH hydroxylase enzymes, under conditions whereby a proline residue on the substrate is hydroxylated, and b) detecting a resultant prolyl hydroxylation of the substrate.

In such assay methods, the mixture may further comprise a candidate agent which modulates the resultant prolyl hydroxylation. In the assay methods the substrate may be any substrate that contains a proline residue that can be hydroxylated. In specific embodiments, such a substrate preferably comprises the sequence LAP*Y, wherein the P* denotes the proline hydroxylated by the hydroxylase. More preferably the substrate comprises a sequence of LAPYI (SEQ ID NO:12). More specifically, peptides that comprise the sequence LAPYIP or LAPYIG are specifically contemplated to be useful substrates for the enzymes described herein Exemplary peptides that may be used include for example, DLDLEMLAPYIPMDDDFQL (SEQ ID NO:11), DLDLEMLAPYIGMDDDFQL (SEQ ID NO:12), DLDLEALAPYIPADDDFQL (SEQ ID NO:10) or DLDLEALAPYIGADDDFQL (SEQ ID NO:13).

The hydroxylase used in the assays described herein preferably is recombinantly expressed in a cell and the detecting step comprises detecting a transcriptional reporter of HIF dependent gene expression.

In particular embodiments, the hydroxylase is an isolated protein, and the detection step comprises detecting a reagent which selectively binds the prolyl hydroxylated substrate.

Another method of the invention relates to screening for a modulator of HIF prolyl hydroxylation, comprising the steps of:

a) incubating a mixture comprising an isolated HIF-specific prolyl hydroxylase (HPH) protein or recombinantly expressed HPH selected from the group consisting of a protein of SEQ ID NO:2, SEQ ID NO:4 and SEQ ID NO: 6, and a peptide substrate of HPH hydroxylase enzymes, under conditions whereby a proline residue on the substrate is hydroxylated, and

b) detecting a resultant prolyl hydroxylation of the substrate;

wherein the incubating step is performed in the presence and absence of candidate modulator of prolyl hydroxylation wherein an increase in the prolyl hydroxylation in the presence of the candidate modulator is indicative of the modulator being an enhancer of prolyl hydroxylation and a decrease in the prolyl hydroxylation in the presence of the candidate modulator is indicative of the modulator being an inhibitor of prolyl hydroxylation.

These and other aspects of the present invention will become apparent upon reference to the following detailed description and attached drawings. All references disclosed herein are hereby incorporated by reference in their entirety as if each was incorporated individually.

### BRIEF DESCRIPTION OF SEVERAL VIEWS OF THE DRAWINGS

FIG. 1. Alignment of rhesus and human HPH sequences performed with Vector NTI. Human and rhesus differences are underlined and denoted above with *(PHD1) (SEQ ID NO:2 for rhesus; SEQ ID NO:7 for human), +(PHD2) (SEQ ID N:4 for rhesus; SEQ ID NO:8 for human), and ̂(PHD3) (SEQ ID NO:6 for rhesus; SEQ ID NO:9 for human).

FIG. 2. Schematic description of the HPH assay used to identify inhibitors or stimulators of HPH.

FIG. 3. Representative data obtained with the assay exemplifying inhibition of HPH1 enzyme with succinate. The y-axis represents the Percent of Control and the x-axis the concentration of succinate (mM).

FIG. 4. Representative data obtained with the assay exemplifying inhibition of HPH2 enzyme with succinate. The y-axis represents the Percent of Control and the x-axis the concentration of succinate (mM).

FIG. 5. Representative data obtained with the assay exemplifying inhibition of HPH3 enzyme with succinate. The y-axis represents the Percent of Control and the x-axis the concentration of succinate (mM).

### DETAILED DESCRIPTION OF THE INVENTION

The present invention relates to sequences for novel HIF-specific propyl hydroxylases (HPH). More particularly, the HPH from rhesus monkeys. The novel HPH nucleic acid sequences may be used to produce HPH proteins that will allow use of these proteins in the identification and testing of inhibitors of HIF HPH for use as therapeutic agents in the treatment of heart disease, tumor vascularization and growth.

HIF1α plays an important role in promoting tumor progression and is overexpressed in common human cancers, including breast, colon, lung, and prostate carcinoma. Overexpression of HIFα is sometimes observed in cancers, such as clear cell renal cell carcinoma, even at normoxia. Mutations that inactivate tumor suppressor genes or activate oncogenes have, as one of their consequences, upregulation of HIF1α activity, either through an increase in HIF1α protein expression, HIF1α transcriptional activity, or both (Semenza, Pediatr. Res., 2001, 49, 614 617). It is known that HIF1α activitity plays a role in heart failure by upregulating cardiac ET1 a gene product involved in heart failure. Abnormal expression or regulation of HIF1α also has been suggested as having a role in preaclampsia and in pulmonary fibrosis and diseases associated with nickel exposure (Andrew et al. Am. J. Physiol Lung Cell Mol. Physiol., 281 L607, 2001). The involvement of HIF1α in malignancies is well documented as is the role of HIF in angiogenesis.

Despite the involvement of HIF and hypoxia responses in various disease states, there are currently significant needs to identify therapeutic agents that effectively inhibit the synthesis or action of HIF-1a.

There are published HPH sequences for human, mouse, rat, dogs, orangutan and chimp. However, the literature at best is confusing regarding the gene/protein nomenclature. HPH genes were originally labeled EGLN (1, 2, 3) for Egg-Laying Mutant Nine (derived from C. elegans gene name) which the protein was referred to as prolyl hydroxylase domain (PHD 2, 1, 3) or HIF-Prolyl Hydroxylase (HPH 1, 2, 3). Additionally, while the names of the proteins were interchangeable, the gene identifier (i.e., the notation of “1” “2” and “3”) has not been consistently employed in the literature. For example, HPH 1, 2, 3 equate with PHD 2, 1 and 3 respectively, and with EGLN 1, 2, and 3, respectively. However, in other instances the literature did not follow this convention.

For purposes of clarity, the HPH sequences defined herein are assigned “1” “2” and “3” designations according to length of the sequences. HPH 1 of the present invention has a nucleic acid sequence of SEQ ID NO: 1 and a protein sequence of SEQ ID NO:2. HPH 2 of the present invention has a nucleic acid sequence of SEQ ID NO:3 and a protein sequence of SEQ ID NO:4. HPH 3 of the present invention has a nucleic acid sequence of SEQ ID NO:5 and a protein sequence of SEQ ID NO:6. The present invention also encompasses expression. constructions, and vectors that are capable of producing proteins of SEQ ID NO: 2, 4 and 6 and methods of recombinant production of such proteins.

### I. Isolated HPH Nucleic Acids

In the discussion presented herein “HPH” refers to the class of proteins and their encoding nucleic acids identified herein, thus “HPH” should be interpreted as “HPH 1, HPH 2 or HPH 3 of the present invention.” One aspect of the invention pertains to isolated nucleic acid molecules that encode HPH proteins or biologically active portions thereof, as well as nucleic acid fragments sufficient for use as hybridization probes to identify these HPH nucleic acid molecules (e.g., HPH mRNA) and fragments for use as PCR primers for the amplification or mutation of HPH nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. In addition, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:3 or SEQ ID NO:5 (for HPH 1, HPH 2 and HPH 3, respectively) or a portion thereof, can be isolated using standard molecular biology techniques and the sequence information provided herein. Using all or a portion of the nucleic acid sequence of SEQ ID NO:1, SEQ ID NO:3 or SEQ ID NO:5, as hybridization probes, HPH nucleic acid molecules can be isolated using standard hybridization and cloning techniques (e.g., as described in Sambrook, J. et al. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, SEQ ID NO:3 or SEQ ID NO:5, can be isolated by the polymerase chain reaction (PCR) using synthetic oligonucleotide primers designed based upon the sequence of SEQ ID NO:1, SEQ ID NO:3 or SEQ ID NO:5.

A nucleic acid of the invention can be amplified using cDNA, mRNA or alternatively, genomic DNA, as a template and appropriate oligonucleotide primers according to standard PCR amplification techniques. The nucleic acid amplified in this manner can be cloned into an appropriate vector and characterized by DNA sequence analysis. As an alternative embodiment, oligonucleotides corresponding to HPH nucleotide sequences can be readily prepared by standard synthetic techniques, e.g., using an automated DNA synthesizer.

The invention also contemplates isolated nucleic acid molecules that are the complement of the nucleotide sequences shown in SEQ ID NO:1, SEQ ID NO:3 or SEQ ID NO:5, or a portion thereof. A nucleic acid molecule which is complementary to the nucleotide sequence shown in SEQ ID NO:1, SEQ ID NO:3 or SEQ ID NO:5, is one which is sufficiently complementary to the nucleotide sequence shown in SEQ ID NO:1, SEQ ID NO:3 or SEQ ID NO:5, such that it can hybridize to the nucleotide sequence shown in SEQ ID NO:1, thereby forming a stable duplex. In a particular embodiment, the complementary sequences of the invention are exact complements of the nucleic acid molecules of the invention, for example, a nucleotide sequence of SEQ ID NO:1, SEQ ID NO:3 or SEQ ID NO:5, a nucleotide sequence encoding a polypeptide of SEQ ID NO:2, SEQ ID NO: 4 or SEQ ID NO: 6 or an allelic variant thereof. For example, the complement may be a full and complete complement of a nucleic acid molecule of the invention, for example, the nucleotide sequence of SEQ ID NO:1, SEQ ID NO:3 or SEQ ID NO:5.

In still another embodiment, an isolated nucleic acid molecule of the invention comprises a nucleotide sequence which is a contiguous nucleic acid sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, 91%, 92%, 93%, 94%, and even more preferably at least about 95%, 96%, 97%, 98%, 99% of the length of the nucleotide sequence of SEQ ID NO:1, SEQ ID NO:3 or SEQ ID NO:5.

In particular, portions of the nucleic acid sequences of SEQ ID NO:1, SEQ ID NO:3. or SEQ ID NO:5 will be used as probes or primers or a fragment encoding a portion of a HPH protein, e.g., a biologically active portion of a HPH protein. The nucleotide sequence determined from the cloning of the HPH gene allows for the generation of probes and primers designed for use in identifying and/or cloning other HPH family members, as well as HPH homologues from other species. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, of an anti-sense sequence of SEQ ID NO:1, SEQ ID NO:3 or SEQ ID NO:5, or of a naturally occurring allelic variant or mutant of SEQ ID NO:1, SEQ ID NO:3 or SEQ ID NO:5.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the HPH nucleotide sequences can be used to detect (e.g., specifically detect) transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. In another embodiment, a set of primers is provided, e.g., primers suitable for use in a PCR, which can be used to amplify a selected region of a HPH sequence, e.g., a domain, region, site or other sequence described herein. The primers should be at least 5, 10, 15, 20, 25, 30, 35, 40, 45 or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which misexpress a HPH protein, such as by measuring a level of a HPH-encoding nucleic acid in a sample of cells from a subject, e.g., detecting HPH mRNA levels or determining whether a genomic HPH gene has been mutated or deleted.

A nucleic acid fragment encoding a “biologically active portion of a HPH protein” can be prepared by isolating a portion of the nucleotide sequence of SEQ ID NO:1, SEQ ID NO:3 or SEQ ID NO:5, which encodes a polypeptide having a HPH biological activity, expressing the encoded portion of the HPH protein (e.g., by recombinant expression in vitro) and assessing the activity of the encoded portion of the HPH protein using standard assay techniques known in the art or those techniques described, for example, in the Examples set forth herein. In an exemplary embodiment, the nucleic acid molecule is at least 50, 100, 150, 200, 250, 300, 350, 400, 450, 500, 550, 600, 650, 700, 750, 1000 or 1250 or more nucleotides in length and encodes a protein having a HPH activity.

The invention further encompasses nucleic acid molecules that differ from the nucleotide sequence shown in SEQ ID NO:1, SEQ ID NO:3 or SEQ ID NO:5, due to degeneracy of the genetic code and thus encode the same HPH proteins as those encoded by the nucleotide sequence shown in SEQ ID NO:1, SEQ ID NO:3 or SEQ ID NO:5. In another embodiment, an isolated nucleic acid molecule of the invention has a nucleotide sequence encoding a protein having an amino acid sequence which differs by at least 1, but no greater than 5, 10, 20, 50 or 100 amino acid residues from the amino acid sequence shown in SEQ ID NO:2, SEQ ID NO:4 or SEQ ID NO:6.

Nucleic acid variants can be naturally occurring, such as allelic variants (same locus), homologues (different locus), and orthologues (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

As used herein, the terms “gene” and “recombinant gene” refer to nucleic acid molecules which include an open reading frame encoding a HPH protein, e.g., oilseed HPH protein, and can further include non-coding regulatory sequences, and introns.

Accordingly, in one embodiment, the invention features isolated nucleic acid molecules which encode a naturally occurring allelic variant of a polypeptide comprising the amino acid sequence of SEQ ID NO:2, SEQ ID NO:4 or SEQ ID NO:6. Moreover, the nucleic acid molecule may hybridize to a complement of a nucleic acid molecule comprising SEQ ID NO:1, SEQ ID NO:3 or SEQ ID NO:5, for example, under stringent hybridization conditions.

In addition to the HPH of SEQ ID NO:1, SEQ ID NO:3 or SEQ ID NO:5, it will be appreciated by those of ordinary skill in the art that DNA sequence polymorphisms that lead to changes in the amino acid sequences of the HPH proteins may exist within a population (e.g., the Rhesus monkey population). Such genetic polymorphism in the HPH gene may exist among individuals within a population due to natural variation. Such natural variations can typically result in 1-5% variance in the nucleotide sequence of the HA gene. Allelic variants of the HPH include both functional and non-functional HPH proteins. Functional allelic variants are naturally occurring amino acid sequence variants of the HPH protein that maintain the ability to, e.g., (i) interact with a HPH substrate or target molecule (e.g., a proline containing peptide); and/or (ii) form a hydroxyl group in a HPH substrate or target molecule. Functional allelic variants will typically contain only a conservative substitution of one or more amino acids of SEQ ID NO:2, SEQ ID NO:4 or SEQ ID NO:6 or a substitution, deletion or insertion of non-critical residues in non-critical regions of the protein.

Non-functional allelic variants are naturally occurring amino acid sequence variants of the HPH protein that do not have the ability to, e.g., (i) interact with a HPH substrate or target molecule (e.g., a peptide that comprises the sequence LAPY, such as for example, DLDLEMLAPYIPMDDDFQL or DLDLEALAPYIPADDDFQL); and/or (ii) form a hydroxyl group in a HPH substrate or target molecule. Non-functional allelic variants will typically contain a non-conservative substitution, a deletion, or insertion, or premature truncation of the amino acid sequence of SEQ ID NO:2, SEQ ID NO:4 or SEQ ID NO:6 or a substitution, insertion, or deletion in critical residues or critical regions of the protein.

Nucleic acid molecules corresponding to natural allelic variants and homologues of the HPH cDNAs of the invention can be isolated based on their homology to the HPH nucleic acids disclosed herein using the cDNAs disclosed herein, or a portion thereof, as a hybridization probe according to standard hybridization techniques under stringent hybridization conditions.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4.times. sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including, but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH2PO4, 7% SDS at about 65° C., followed by one or more washes at 0.02M NaH2PO4, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule of the invention that hybridizes under stringent conditions to the sequence of SEQ ID NO:1, SEQ ID NO:3 or SEQ ID NO:5 correspond to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the HPH sequences that may exist in the population, the skilled artisan will further appreciate that changes can be introduced by mutation into the nucleotide sequences of SEQ ID NO:1, SEQ ID NO:3 or SEQ ID NO:5, thereby leading to changes in the amino acid sequence of the encoded HPH proteins, without altering the functional ability of the HPH proteins. For example, nucleotide substitutions leading to amino acid substitutions at “non-essential” amino acid residues can be made in the sequence of SEQ ID NO:1, SEQ ID NO:3 or SEQ ID NO:5. A “non-essential” amino acid residue is a residue that can be altered from the wild-type sequence of Macaca mulatta HPH (e.g., the sequence of SEQ ID NO:2, SEQ ID NO:4, SEQ ID NO:6) without altering the biological activity, whereas an “essential” amino acid residue is required for biological activity. For example, amino acid residues that are conserved between the HPH proteins of the present invention and other members of the fatty acid HPH family are not likely to be amenable to alteration.

Accordingly, another aspect of the invention pertains to nucleic acid molecules encoding HPH proteins that contain changes in amino acid residues that are not essential for activity. Such HPH proteins differ in amino acid sequence from SEQ ID NO:2, SEQ ID NO:4 or SEQ ID N:6 yet retain biological activity. In one embodiment, the isolated nucleic acid molecule comprises a nucleotide sequence encoding a protein, wherein the protein comprises an amino acid sequence that has a contiguous length of least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical to SEQ ID NO:2, SEQ ID NO: 4 or SEQ ID NO:6 e.g., to the entire length of SEQ ID NO:2, SEQ ID NO: 4 or SEQ ID NO:6.

An isolated nucleic acid molecule encoding a HPH protein homologous to the protein of SEQ ID NO:2, SEQ ID NO: 4 or SEQ ID NO:6 can be created by introducing one or more nucleotide substitutions, additions or deletions into the nucleotide sequence of SEQ ID NO:1, SEQ ID NO:3 or SEQ ID NO:5, such that one or more amino acid substitutions, additions or deletions are introduced into the encoded protein. Mutations can be introduced into SEQ ID NO:1, SEQ ID NO:3 or SEQ ID NO:5 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, typtophan, histidine). Thus, a predicted nonessential amino acid residue in a HPH protein is preferably replaced with another amino acid residue from the same side chain family. Alternatively, in another embodiment, mutations can be introduced randomly along all or part of a HPH coding sequence, such as by saturation mutagenesis, and the resultant mutants can be screened for HPH biological activity to identify mutants that retain activity. Following mutagenesis of SEQ ID NO:1, SEQ ID NO:3 or SEQ ID NO:5, the encoded protein can be expressed recombinantly and the activity of the protein can be determined.

In a preferred embodiment, a mutant HPH protein can be assayed for the ability to (i) interact with a HPH substrate or target molecule (e.g., a peptide that comprises the sequence LAPY, such as for example, DLDLEMLAPYIPMDDDFQL or DLDLEALAPYIPADDDFQL) and/or (ii) form a hydroxyl group in a HPH substrate or target molecule using standard assays known in the art or those assays described herein.

### II. Isolated HPH Proteins

A further aspect of the invention describes isolated or recombinant HPH proteins and polypeptides, and biologically active portions thereof that have a sequence of SEQ ID NO:2, SEQ ID NO:4 or SEQ ID NO:6. In one embodiment, native HPH proteins can be isolated from M. mulatta cells or tissue sources by an appropriate purification scheme using standard protein purification techniques. In another embodiment, HPH proteins are produced by recombinant DNA techniques. Alternative to recombinant expression, a HPH protein or polypeptide can be synthesized chermically using standard peptide synthesis techniques.

An “isolated” or “purified” protein or biologically active portion thereof is substantially free of cellular material or other contaminating proteins from the cell or tissue source from which the HPH protein is derived, or substantially free from chemical precursors or other chemicals when chemically synthesized. The language “substantially free of cellular material” includes preparations of HPH protein in which the protein is separated from cellular components of the cells from which it is isolated or recombinantly produced. In one embodiment, the language “substantially free of cellular material” includes preparations of HPH protein having less than about 80%, 70%, 60%, 50%, 40%, or 30% (by dry weight) of non-HPH protein (also referred to herein as a “contaminating protein”), more preferably less than about 20% of non-HPH protein, still more preferably less than about 10% of non-HPH protein, and most preferably less than about 5% non-HPH protein. When the HPH protein or biologically active portion thereof is recombinantly produced, it is also preferably substantially free of culture medium, i.e., culture medium represents less than about 20%, more preferably less than about 10%, and most preferably less than about 5% of the volume of the protein preparation.

As used herein, a “biologically active portion” of a HPH protein includes a fragment of a HPH protein which participates in an interaction between a HPH molecule and a non-HPH molecule (e.g., a HPH substrate such as peptide containing a prolyl residue amenable to hydroxylation). Biologically active portions of a HPH protein include peptides comprising amino acid sequences sufficiently homologous to or derived from the HPH amino acid sequences, e.g., the amino acid sequences shown in SEQ ID NO:2, SEQ ID NO:4, SEQ ID NO:6 which include sufficient amino acid residues to exhibit at least one activity of a HPH protein. Typically, biologically active portions comprise a domain or motif with at least one activity of the HPH protein, for example, the ability to (i) interact with a HPH substrate or target molecule (e.g., a peptide containing a proline residue for hydroxylation) and/or (ii) form a hydroxyl group in a HPH substrate or target molecule, a biologically active portion of a HPH protein can be a polypeptide which is, for example, 10, 15, 20, 25, 30, 35, 40, 45, 50, 75, 100, 125, 150, 175, 200, 250, 300, 350, 400 or more amino acids in length.

In a preferred embodiment, a HPH protein has an amino acid sequence shown in SEQ ID NO:2, SEQ ID NO:4, or SEQ ID NO:6. In other embodiments, the HPH protein is substantially identical to SEQ ID NO:2, SEQ ID NO:4 or SEQ ID NO:6 and retains the functional activity of the protein of SEQ ID NO:2, SEQ ID NO:4 or SEQ ID NO:6, yet differs in amino acid sequence due to natural allelic variation or mutagenesis, as described in detail in subsection I above. In another embodiment, the HPH protein is a protein which comprises an amino acid sequence that contains a contiguous length that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, 91%, 92%, 93%, 94%, and even more preferably at least about 95%, 96%, 97%, 98%, 99% or more identical to SEQ ID NO:2, SEQ ID NO:4 or SEQ ID NO:6.

In another embodiment, the invention features a HPH protein which is encoded by a nucleic acid molecule consisting of a nucleotide sequence of a length that is least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, 91%, 92%, 93, 94%, and even more preferably at least about 95%, 96%, 97%, 98%, 99% or more identical to a contiguous nucleotide sequence of SEQ ID NO:1, SEQ ID NO:3 or SEQ ID NO:5, or a complement thereof. This invention further features a HPH protein which is encoded by a nucleic acid molecule consisting of a nucleotide sequence which hybridizes under stringent hybridization conditions to a complement of a nucleic acid molecule comprising the nucleotide sequence of SEQ ID NO:1, SEQ ID NO:3 or SEQ ID NO:5.

### III. Methods of Producing HPH

The present invention provides new and improved methods of producing hydroxylating proline-containing peptides.

A. Recombinant Cells and Methods for Culturing Cells

The present invention further features recombinant vectors that include nucleic acid sequences that encode the gene products as described herein, preferably HPH gene products of sequence SEQ ID NO:2, SEQ ID NO:4 or SEQ ID NO:6 or fragments thereof. The term recombinant vector includes a vector (e.g., plasmid) that has been altered, modified or engineered such that it contains greater, fewer or different nucleic acid sequences than those included in the native vector or plasmid. In one embodiment, a recombinant vector includes the nucleic acid sequence encoding at least one HPH enzyme operably linked to regulatory sequences. The phrase “operably linked to regulatory sequence(s)” means that the nucleotide sequence of interest is linked to the regulatory sequence(s) in a manner which allows for expression (e.g., enhanced, increased, constitutive, basal, attenuated, decreased or repressed expression) of the nucleotide sequence, preferably expression of a gene product encoded by the nucleotide sequence (e.g., when the recombinant vector is introduced into a cell).

The term “regulatory sequence” includes nucleic acid sequences which affect (e.g., modulate or regulate) expression of other (non-regulatory) nucleic acid sequences. In one embodiment, a regulatory sequence is included in a recombinant vector in a similar or identical position and/or orientation relative to a particular gene of interest as is observed for the regulatory sequence and gene of interest as it appears in nature, e.g., in a native position and/or orientation. For example, a gene of interest (e.g., a M. mulatta HPH gene of SEQ ID NO: 1, SEQ ID NO:3 or SEQ ID NO:5) can be included in a recombinant vector operably linked to a regulatory sequence which accompanies or is adjacent to the gene in the natural organism (e.g., operably linked to “native” regulatory sequence such as the “native” fatty acid HPH promoter). Alternatively, a gene of interest (e.g., a M. mulatta HPH gene of SEQ ID NO: 1, SEQ ID NO:3 or SEQ ID NO:5) can be included in a recombinant vector operably linked to a regulatory sequence which accompanies or is adjacent to another (e.g., a different) gene in the natural organism. For example, HPH gene can be included in a vector operably linked to non-HPH regulatory sequences. Alternatively, a gene of interest (e.g., a M. mulatta HPH gene of SEQ ID NO: 1, SEQ ID NO:3 or SEQ ID NO:5) can be included in a vector operably linked to a regulatory sequence from another organism. For example, regulatory sequences from other microbes (e.g., other bacterial regulatory sequences, bacteriophage regulatory sequences and the like) can be operably linked to a particular gene of interest.

Preferred regulatory sequences include promoters, enhancers, termination signals and other expression control elements (e.g., binding sites for transcriptional and/or translational regulatory proteins, for example, in the transcribed mRNA). Such regulatory sequences are described, for example, in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Regulatory sequences include those which direct constitutive expression of a nucleotide sequence in a cell (e.g., constitutive promoters and strong constitutive promoters), those which direct inducible expression of a nucleotide sequence in a cell (e.g., inducible promoters, for example, xylose inducible promoters) and those which attenuate or repress expression of a nucleotide sequence in a cell (e.g., attenuation signals or repressor sequences). It is also within the scope of the present invention to regulate expression of a gene of interest by removing or deleting regulatory sequences. For example, sequences involved in the negative regulation of transcription can be removed such that expression of a gene of interest is enhanced.

In one embodiment, a recombinant vector of the present invention includes nucleic acid sequences that encode at least one gene product (e.g., a M. mulatta HPH protein of SEQ ID NO: 2, SEQ ID NO:4 or SEQ ID NO:6) operably linked to a promoter or promoter sequence.

In a particular embodiment, a constitutive promoter such as the CMV promoter is used. Other promoters that may be used include by are not limited to but are not limited to, the SV40 early promoter region; RSV or other retroviral LTRs; herpes thymidine kinase promoter, human cytomegalovirus (CMV) immediate early promoter/enhancer. Other promoters that have been used for this purpose include the elastase 1 gene control region; insulin gene control region; immunoglobulin gene control region; mouse mammary tumor virus control region; albumin gene control region; alpha-fetoprotein gene control region; alpha 1-antitrypsin gene control region and beta-globin gene control region.

In yet another embodiment, a recombinant vector of the present invention includes a terminator sequence or terminator sequences (e.g., transcription terminator sequences). The term “terminator sequences” includes regulatory sequences which serve to terminate transcription of mRNA. Terminator sequences (or tandem transcription terminators) can further serve to stabilize mRNA (e.g., by adding structure to mRNA), for example, against nucleases.

In yet another embodiment, a recombinant vector of the present invention includes antibiotic resistance sequences. The term “antibiotic resistance sequences” includes sequences which promote or confer resistance to antibiotics on the host organism. In one embodiment, the antibiotic resistance sequences are selected from the group consisting of cat (chloramphenicol resistance), tet (tetracycline resistance) sequences, erm (erythromycin resistance) sequences, neo (neomycin resistance) sequences and spec (spectinomycin resistance) sequences. Recombinant vectors of the present invention can further include homologous recombination sequences (e.g., sequences designed to allow recombination of the gene of interest into the chromosome of the host organism). For example, amyE sequences can be used as homology targets for recombination into the host chromosome.

The term “manipulated cell” includes a cell that has been engineered (e.g., genetically engineered) or modified such that the cell expresses and preferably secretes at least HPH protein of the invention (e.g., a protein of SEQ ID NO:2; SEQ ID NO:4 or SEQ ID NO:6). Recombinant cells are included as manipulated cells. The recombinant host cell that is engineered to express the HPH protein of the invention may be any cell typically used for protein production. DNA sequences encoding the HPH proteins can be expressed in vitro by DNA transfer into a suitable host cell. “Host cells” are cells in which a vector can be propagated and its DNA expressed. Host cells include both prokaryotic and eukaryotic cells. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

Examples of suitable host cells that may be transformed using the nucleic acids of the present invention include preferably are mammalian cells, such as for example cells of an established mammalian cell line, including, without limitation, CHO (e.g., ATCC CCL 61), COS-1 (e.g., ATCC CRL 1650), baby hamster kidney (BHK) and HEK293 (e.g., ATCC CRL 1573; Graham et al., J. Gen. Virol. 36:59-72, 1977) cell lines. Other suitable cell lines include, without limitation, Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1) and DUKX cells (Urlaub and Chasin, Proc. Natl. Acad. Sci. USA 77:4216-4220, 1980). Also useful are 3T3 cells, Namalwa cells, myelomas and fusions of myelomas with other cells.

Modification or engineering recombinant cells can be according to any methodology described herein including, but not limited to, deregulation of a biosynthetic pathway and/or overexpression of at least one biosynthetic enzyme. A “manipulated” enzyme (e.g., a “manipulated” biosynthetic enzyme) includes an enzyme, the expression or production of which has been altered or modified such that at least one upstream or downstream precursor, substrate or product of the enzyme is altered or modified, for example, as compared to a corresponding wild-type or naturally occurring enzyme.

### IV. HPH-Based Assays

The HPH sequences identified herein may be used in various methods and as compositions for further development. For example, the HPH sequences identified in the present invention will be useful in assays for identification of drugs that can be used to modulate prolyl hydroxylation. In general, such assays are used to identify or confirm a modulator's specific biochemical or molecular effect with respect to the activity of HPH protein.

In this manner, a particular use of the sequences of the present invention provides efficient methods of identifying agents, compounds or lead compounds for agents which modulate HPH activity. The sequences identified may be used in automated, cost-effective high throughput screening methods for screening of chemical libraries for lead compounds that may be used as inhibitors of HPH. Agent identified by such screening methods will be useful and used in the pharmaceutical industries for animal and human trials; for example, the reagents may be derivatized and rescreened in assays to optimize activity and minimize toxicity for pharmaceutical development.

The compositions of the invention may be used in any screening assay that uses HPH. For example, assays may comprise the steps of: a) incubating a mixture comprising an isolated or recombinantly expressed HPH and a substrate of the hydroxylase, under conditions whereby the hydroxylase hydroxylates prolyl residues on the substrate, and b) detecting a resultant prolyl hydroxylation of the substrate.

In the screening assay embodiment, the screening method comprises contacting a suitable mixture that comprises an HPH polypeptide or a recombinant cell expressing. HPH-encoding nucleic acid with a candidate agent under conditions whereby, but for the presence of the agent, the system provides a reference HPH activity (e.g. hydroxylation of a proline containing peptide such as those described herein). A statistically significant difference between the agent-biased HPH activity and the reference activity indicates that the candidate agent modulates prolyl hydroxylation activity, and hence may be used as a modulator of HIF1α activity. The HPH polypeptide or recombinant cell expressing HPH-encoding nucleic acid used in the assay may comprise any of the nucleic acids or polypeptides described above.

A. HPH Components

Exemplary HPH sequences for use in these embodiments include HPH 1, HPH 2 and HPH 3 and variants and homologs thereof. HPH 1 of the present invention has a nucleic acid sequence of SEQ ID NO: 1 and a protein sequence of SEQ ID NO:2. HPH 2 of the present invention has a nucleic acid sequence of SEQ ID NO:3 and a protein sequence of SEQ ID NO:4. HPH 3 of the present invention has a nucleic acid sequence of SEQ ID NO:5 and a protein sequence of SEQ ID NO:6. The assays will use at least the prolyl hydroxylase domain of the HPH proteins described herein. While the native, full-length proteins provide the best model for drugs screens, one may also use truncations of HPH 1, HPH 2 and HPH 3 which retain HPH activity (HIF prolyl hydroxylase domains) of the proteins of SEQ ID NO 2, SEQ ID NO:4 and SEQ ID NO:6, respectively, which may optionally be coupled to additional homologous (corresponding HPH) or heterologous residues, i.e. the domain may be part of a fusion product with another peptide or polypeptide, e.g. a tag for detection or anchoring, etc.

For production methods the HPH enzymes may be expressed in e.g., bacterial cells such that they could be purified by affinity chromatography as soluble proteins. In exemplary embodiments, heterologous expression of these proteins was performed by subcloning the genes into pvl1393 vectors with either N- or C-terminal Glutathione S-Transferase (GST) tags to aid in purification of the proteins. SF9 cells were co-transfected with BaculoGold™ Linearized Baculovirus DNA (Becton Dickinson) and the complementing pvl1393 transfer vector containing the gene of interest. To identify recombinant virions, a plaque purification was completed and viral amplifications from one isolated plaque were done in SF9 cells. After two viral amplifications, sufficient amounts of a high titered pure virus stock were generated for large scale protein production. Expression was verified using an anti-GST antibodies at each step of the viral amplification process.

B. Substrates

The assays will employ a substrate that contains a prolyl residue to be hydroxylated by the recited hydroxylases of the invention. Suitable peptide substrates are readily identified in the subject prolyl hydroxylase screening assays. Any peptide that contains random proline residue amenable to hydroxylation may be used as a substrate in the screening assays. Preferably, however, the peptide may be derived from a HIF 1α oxygen degradation domain (ODD) peptide comprising Pro564 which is the natural substrate for HPH (see, experimental, below, and Jaakkola et al. and Ivan et al., below).

Thus, the prolyl hydroxylation substrate for use in the assay described herein may be any substrate routinely used in determining HIF-1α hydroxylation by the HPH enzymes. Exemplary substrates are described in e.g., Huang et al (“Sequence Determinants in Hypoxia-inducible Factor-1α for Hydroxylation by the Prolyl Hydroxylases PHD1, PHD2, and PHD3” J. Biol. Chem., Vol. 277, No. 42, Issue of October 18, pp. 39792-39800, 2002). As noted therein, HPH enzymes hydroxylate specific prolines in HIF subunits in the context of a strongly conserved LXXLAP sequence motif (where X indicates any amino acid and P indicates the hydroxyl acceptor proline). It is noted that in substrates for an HPH assay that comprise a sequence of LXXLAP mutations can be readily tolerated at the −5, −2, and −1 positions (relative to proline) of the LXXLAP motif.

Exemplary peptides that can serve as substrates include peptides of that comprise the sequence LAPY. The peptides may be of any length and any sequence as long as the sequence and length of the peptide renders them readily amenable to prolyl hydroxylation. For example, it has been described that a double Met to Ala substitution in a human HIF-1α ODD domain provide an equivalent HDH substrate with less oxidative reactivity and hence, improved compatibility with mass spectroscopy-based analysis. In general, preferred substrates comprise the peptide LAPY, more preferably LAPYI (SEQ ID NO:12), more preferably LAPYI, wherein the I is coupled to an additional residue, preferably P or G. Thus, peptides that comprise the sequence LAPYIP or LAPYIG are specifically contemplated to be useful substrates for the enzymes described herein Exemplary peptides that may be used include for example, DLDLEMLAPYIPMDDDFQL (SEQ ID NO:11), DLDLEMLAPYIGMDDDFQL (SEQ ID NO:12), DLDLEALAPYIPADDDFQL (SEQ ID NO:10) or DLDLEALAPYIGADDDFQL (SEQ ID NO:13).

The substrates also may be labeled to facilitate detection. Such detectable labels include but are not limited to directly detectable labels e.g., detectable (e.g. radiolabels, fluorescent labels, etc.) or indirectly detectable (e.g. epitope tags, biotin, etc.). Detection of such a label or marker, such as any of those radioactive, fluorescent, biological and enzymatic tags are have been described in the art: see U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Exemplary radiolabels include paramagnetic ions, by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), and also radioisotopes such as astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, 59iron, ³²phosphorus, rhenium¹⁸⁶, rhenium¹⁸⁸, ⁷⁵selenium, ³⁵sulphur, technicium^{99m} and yttrium⁹⁰.

In additional embodiments, the screening assays may further comprise comprises a candidate agent which modulates the resultant prolyl hydroxylation, wherein an agent-biased prolyl hydroxylation is detected. Candidate agents encompass numerous chemical classes, though typically they are organic compounds; preferably small organic compounds and are obtained from a wide variety of sources including libraries of synthetic or natural compounds. A variety of other reagents may also be included in the mixture. These include reagents like cofactors (e.g. Fe(II)), cosubstrates (e.g. dioxygen), salts, buffers, neutral proteins, e.g. albumin, detergents, protease inhibitors, antimicrobial agents, etc.

The HPH compositions of the present invention also may be used in assays that employ known modulators for a variety of purposes, such as comparative or competitive analysis. For example, dimethyl-oxalylglycine is a competitive inhibitor by virtue of its similarity to the cosubstrate, 2-oxoglutarate. Similarly, ascorbate is shown to promote the reaction, and a variety of analogs of 2-oxaglutarate or ascorbate inhibit assay prolyl hydroxylation. In addition, divalent metal ions such as Co2+ can compete with Fe2+ for occupancy and inhibit hydroxylation and iron chelators like deferoxamine mesylate inhibit hydroxylation by competing for Fe2+. Finally, suicide inhibitors are readily constructed by incorporating oxaproline residue into the hydroxylase substrate, e.g. Wu et al., 1999, J Am Chem Soc 121, 587-588.

C. Candidate Substances

The candidate substances used in the screening assays may be small molecule modulators. The screening assays are used to identify these candidate modulators may be cell-based or may use a cell-free system that recreates or retains the HPH activity of protein. As used herein the term “cell-based” refers to assays using live cells, dead cells, or a particular cellular fraction, such as a membrane, endoplasmic reticulum, or mitochondrial fraction. The term “cell free assay” relates to assays using substantially purified protein (either endogenous or recombinantly produced), partially purified or crude cellular extracts. The screening assays detect the HPH activity using a variety of detection methods including fluorescent, radioactive, calorimetric, spectrophotometric, and amperometric methods, to provide a read-out for the prolyl hydroxylation.

The candidate modulators can be any small molecule compound, or a biological entity, such as a protein, sugar, nucleic acid or lipid. Typically, test compounds will be small chemical molecules and peptides. Essentially, any chemical compound can be used as a potential HPH agonist or antagonist, although most often compounds that can be dissolved in aqueous or organic (especially DMSO-based) solutions are used. In general, assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

Preparation and screening of combinatorial chemical libraries is well known to those of skill in the art. Such combinatorial chemical libraries include, but are not limited to, peptide libraries (see, e.g., U.S. Pat. No. 5,010,175, Furka, 1991, and Houghton et al., 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., 1993), vinylogous polypeptides, nonpeptidal peptidomimetics with glucose scaffolding, analogous organic syntheses of small compound libraries, oligocarbamates, and/or peptidyl phosphonates, nucleic acid libraries, peptide nucleic acid libraries (see, e.g. U.S. Pat. No. 5,539,083), antibody libraries (see, e.g., Vaughn et al., 1996, and PCT/US96/10287), carbohydrate libraries (see, e.g., Liang et al., 1996, and U.S. Pat. No. 5,593,853), small organic molecule libraries (see, e.g., benzodiazepines; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 NPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Using lead compounds to help develop improved compounds is know as “rational drug design” and includes not only comparisons with know inhibitors and activators, but predictions relating to the structure of target molecules. The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches. It also is possible to use antibodies to ascertain the structure of a target compound activator or inhibitor. In principle, this approach yields a pharmacophore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

D. Assay Formats

In a distinct embodiment, one may examine the interaction between HPH and its substrate in the presence and absence of the candidate modulator. This interaction may involve a cell free system, i.e., a simple binding assay (filter-based, affinity column, gel exclusion, gel-shift assay), but it may also advantageously incorporate a cell-based system with one or both of HPH, its substrate and the candidate substance being expressed (for example, on the cell surface) by the cell. The cell may naturally express these molecules, or may have been engineered to express or overexpress the molecules. In addition, HPH or the HPH substrate may be a variant that includes moieties that facilitate identification, such as epitopes that are recognized by antibodies, 6×-His tags. Alternatively, the HPH or the HPH substrate may include a label (fluorescent, chemiluminescent, dyes, enzymes).

The assays may be cell-based, such as wherein the hydroxylase is recombinantly expressed in the cell as described above and the detecting step comprises detecting a transcriptional reporter of HIF dependent gene expression. Alternatively, the assay may be run in vitro, wherein the hydroxylase is isolated and preferably provided in a predetermined amount. For in vitro assays, hydroxylation may be detected directly or indirectly. For example, hydroxylation may be directly detected in mass spectroscopy-based assays. Alternatively, the hydroxylation may be detected with a reagent which selectively binds the prolyl hydroxylated substrate, wherein depending on the reagent, the binding may be detected by changes in fluorescent polarization, fluorescence, radiation, catalytic product (e.g. luciferase, galactosidase, etc.), etc. An exemplary direct and continuous assay for determining prolyl hydroxylation is described by Gorres and Raines (Anal Biochem. Mar. 15, 2009;386(2):181-5) and may be readily employed with the HPH 1, 2, or 3 of the present invention.

To identify a modulator, one generally will determine the activity of the HPH in hydroxylating a prolyl moiety in a target substrate in the presence and absence of the candidate substance, a modulator defined as any substance that alters the prolyl hydroxylation function of the HPH. For example, a method generally comprises: (a) providing a prolyl containing peptide substrate; (b) admixing the substrate with a HPH protein preparation; or recombinant cell comprising the HPH of the invention and; (c) measuring the prolyl hydroxylation activity; and (d) comparing the effect(s) measured in step (c) with the effect(s) in the presence of a candidate modulator wherein a difference between the measured effects in (c) and (d) indicates that the candidate is, indeed, a modulator of the HPH activity.

In any of the herein-described assays, a modulation of 5%, 10%, 20%, 30%, 40%, 50%, 60%, 70%, 80%, 90%, 95%, or greater modulation in any detectable characteristic, as described above, is contemplated to be a useful modulation.

It will, of course, be understood that all the screening methods of the present invention are useful in themselves notwithstanding the fact that effective candidates may not be found. The invention provides methods for screening for such candidates, not solely methods of finding them.

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, filters, plates, chambers, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, such as a filter or column, or expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding. Such formats could be advantageously applied to the examination of HPH/HIF1α interactions.

A technique for high throughput screening of compounds is described in WO 84/03564. Large numbers of small peptide test compounds are synthesized on a solid substrate, such as plastic pins or some other surface. Bound polypeptide is detected by various methods.

The activity of recombinant HPH protein may be stimulated in the assays by the addition of ascorbate, 2-oxoglutarate and FeSO4. Thus, screening assays designed to use the HPH compositions of the present invention may preferably include such reagents. As it is known that CoCl2 induces the hypoxic response pathway by stabilizing HIF under normoxic conditions, possibly by competing with Fe2+ for occupancy within the active site of HPH, the screening assays may be performed in the presence and absence of CoCl2 in order to assess the likelihood that the candidate substance being tested will be able to overcome a hypoxic effect.

Substrate specificity of the HPH enzymes of the present invention may be assessed using assays previously used for human HPH enzymes. Previous studies have indicated that amino acids in close proximity to the proline residue targeted for hydroxylation (Pro564 in human HIF-1a) influence HIF-1a modification/pVHL binding. Specifically, mutation of Leu562 to Ala (M. Ivan et al., Science 292, 464 (2001)), Ala563 to Gly (F. Yu, S. B. White, F. S. Lee, Proc. Natl. Acad. Sci. U.S.A. 98, 9630 (2001)) and Tyr565 to Ala (P. Jaakkola et al., Science 292, 468 (2001)) have been shown to prevent prolyl hydroxylation by the endogenous HIF prolyl hydroxylase activity present in cellular extracts. Conversely, mutation of Pro567 to Gly has been shown to exert a slight stimulatory effect in pVHL pull-down assays (P. Jaakkola et al., Science 292, 468 (2001)). In order to examine the substrate specificity of the HPH enzymes of the present invention, individual biotinylated peptide substrates that contain the above point mutations can be used to determine whether the HPH enzymes of the present invention are comparable and therefore serve as appropriate surrogates for human HPH enzymes. The assay also may be performed using each substrate with the Pro564 already hydroxylated allowing one to differentiate the affects of these mutations on pVHL binding from their affects on proline hydroxylation. Wild-type human HPH is unable to modify peptides containing the L562A, A563G or Y565A mutations but does modify peptides containing a mutation of Pro567 to glycine. The substrate specificity of the HPH 1, 2 and 3 enzymes of the present invention can be compared with the above reported substrate specificity of wild-type human HIF prolyl hydroxylase and thus identify the HPH 1, 2, and 3 enzymes of the presented invention as appropriate surrogates for endogenous HIF prolyl hydroxylase.

Assays also are established to demonstrate whether the HPH 1, 2 and 3 enzymes of the invention represent part of the hypoxic response pathway. For example, an appropriate host cell, e.g., human embryonic kidney 293 cells; CHO cells, 3T3 cells, HeLa, HT1080, NHF, LS174, CAL51, 293, CCL39, and GSK3 or any other cell that can be used for recombinant production of proteins is co-transfected with a hypoxia-responsive luciferase reporter (R. K. Bruick, Proc. Natl. Acad. Sci. U.S.A. 97, 9082 (2000)) and increasing amounts of a vector expressing human HIF-1α under the control of the constitutive CMV promoter. Forced overexpression of HIF-1α can overcome the degradation pathway, resulting in accumulation of the HIF transcription factor under normoxic conditions and subsequent induction of the HRE-containing HIF reporter gene (R. K. Bruick, Proc. Natl. Acad. Sci. U.S.A. 97, 9082 (2000)).

Cell-based screening assays usually require systems for recombinant expression of the HPH and may also recombinantly express the substrate of the assay (e.g., a protein substrate containing an amino acid sequence that is amenable to prolyl hydroxylation). Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide other methods for determining protein-protein interactions and elucidation of protein complexes. In certain applications, when HPH-interacting proteins are used in screens to identify small molecule modulators, the binding specificity of the substrate protein containing the prolyl residue to be hydroxylated to the HPH proteins of the invention may be assayed by various known methods such as substrate processing (e.g. ability of the candidate HPH-specific binding agents to function as negative effectors in HPH-expressing cells), and binding equilibrium constants (usually at least about 107 M-1, preferably at least about 108 M-1, more preferably.at least about 109 M-1). For enzymes and receptors, binding may be assayed by, respectively, substrate and ligand processing.

The screening assay may measure a candidate agent's ability to specifically bind to or modulate activity of an HPH polypeptide, a fusion protein thereof, or to cells or membranes bearing the polypeptide or fusion protein. The HPH polypeptide can be full length or a fragment thereof that retains functional HPH activity. The HPH polypeptide may be fused to another polypeptide, such as a peptide tag for detection or anchoring, or to another tag. In a preferred embodiment, the screening assay detects candidate agent-based modulation of HPH hydroxylation and/or binding of to a prolyl containing target peptide, and can be used to assess normal ACAC gene function.

Suitable assay formats that may be adapted to screen for HPH modulators are known in the art. Preferred screening assays are high throughput or ultra high throughput and thus provide automated, cost-effective means of screening compound libraries for lead compounds (Fernandes P B, Curr Opin Chem Biol (1998) 2:597-603; Sundberg S A, Curr Opin Biotechnol 2000, 11:47-53). In one preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, supra; Hertzberg R P and Pope A J, Curr Opin Chem Biol (2000) 4:445-451).

HIF-1 is upregulated in various cells following exposure to hypoxia in vitro. Under hypoxic conditions, HIF-1 stimulates the expression of genes known to be important in tumour cell survival, such as those encoding glycolytic enzymes and VEGF. Induction of such genes by hypoxic conditions may be assayed by growing cells transfected with HPH in hypoxic conditions (such as with 0.1% O2, 5% CO2, and balance N2, generated in a Napco 7001 incubator (Precision Scientific)) and normoxic conditions, followed by assessment of gene activity or expression by Taqman®. For example, a hypoxic induction assay system may comprise a cell that expresses an HPH. A test agent can be added to the hypoxic induction assay system and changes in hypoxic response relative to controls where no test agent is added, identify candidate HPH modulating agents. In some embodiments of the invention, the hypoxic induction assay may be used as a secondary assay to test a candidate modulating agents that is initially identified using a peptide based in vitro assay. A hypoxic induction assay may thus be used to test whether the candidate modulator will have a direct role in the hypoxic response. For example, a hypoxic induction assay may be performed on cells that express HPH and the differences in hypoxic response of the cells in the presence and absence of the candidate modulator will allow determination of the type and degree of effect of the candidate on hypoxic induction.

The recombinant cells can be used to determine the effect of the candidate modulators in inhibiting or enhancing HPH gene expression, preferably mRNA expression. In general, expression analysis comprises comparing HPH expression in like populations of cells (e.g., two pools of cells that recombinantly express the HPH of the invention) in the presence and absence of the modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that HPH mRNA expression is reduced in cells treated with the modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the HPH protein or specific peptides. A variety of means including Western blotting, ELISA, or in situ detection, are available (Harlow E and Lane D, 1988 and 1999, supra). In some cases, screening assays described for small molecule modulators, particularly in assay systems that involve HPH mRNA expression, may also be used to test nucleic acid modulators.

A useful tool for examining gene expression, as discussed above, is a nucleic acid array. Such arrays are commercially available from a variety of sources (e.g., Affymetrix) and involve a plurality of nucleic acid sequences fixed in a particular fashion on the surface of a support such as a chip or wafer. The plurality of nucleic acids sequences represents a plurality of different target genes, and hybridization of mRNA from a target cell to the array, followed by detection, can demonstrate expression of a target gene as well as relative amounts.

In cell based assays the candidate modulators are typically added to the media. In assays employing isolated protein, the candidate modulators are added to the reaction mixture.

The HPH-based compositions of the invention also include systems and kits for the disclosed HIF-specific prolyl hydroxylation assays. For in vitro assays, for example, such systems and kits can include predetermined amounts of the isolated HPH and of one or more suitable substrates. The systems and kits will generally also comprise further reagents described herein to facilitate the reaction, suitable packaging, and written instructions describing the hydroxylase, the substrate and the assay.

E. Exemplary Screening Assay Outputs

In exemplary embodiments to determine whether the HPH enzymes of the invention have activity relating to HIF-1a-directed prolyl hydroxylase activity, the nucleic acid encoding the HPH may be cloned into an appropriate expression vector. An exemplary such expression vector is pcDNA3.1/V5-His expression vector (Invitrogen). The putative HPH enzymes are then in vitro transcribed/translated in cell, e.g., a rabbit reticulocyte lysate system (available from Promega). In an exemplary such assay, the candidate enzymes are synthesized according to manufacturer's instructions in the TNT Coupled Reticulocyte Lysate System (Promega) for 1 hr at 30° C. Gene expression can be confirmed by Western blot analysis using an antibody specific for the carboxy-terminal V5 tag. For example 12.5 μl of each in vitro transcription/translation reaction is incubated for 30 min at 30° C. in a reaction buffer containing 20 mM Tris-Cl (pH 7.5), 5 mM KCl, 1.5 mM MgCl2, 1 mM DTT, 2 mM 2-oxoglutarate, 2 mM ascorbate and 250 μM FeSO4 in the presence of 30 μl ImunoPure Immobilized Streptavidin beads that had previously been incubated with 1 μg of substrate peptide for 30 min at room temperature and washed to remove excess peptide. Following incubation, the beads are washed with 1 ml cold buffer (20 mM Tris-Cl (pH 8.0), 100 mM NaCl, 1 mM EDTA and 0.5% NP-40) and incubated for 10 min at 4° C. with approximately 35 kcpm of [35 S]-labeled human VHL in 500 μl EBC buffer (50 mM Tris-Cl (pH 8.0), 120 mM NaCl, 0.5% NP-40). The beads are then washed cold NTEN buffer and bound [35 S] is measured by scintillation counting. [35 S]-labeled human VHL can be readily prepared by cloning human VHL cDNA into the pcDNA3.1/V5-HIS vector (Invitrogen Corp.) using the TNT Coupled Reticulocyte Lysate System (Promega) and [35 S]-L-Met (Amersham Pharmacia Biotech) and desalted using a PD-10 column (Amersham Pharmacia Biotech).

Prolyl hydroxylase activity is measured by incubating the translation products in the presence of ascorbate, 2-oxoglutarate, and FeSO4 with a biotinylated peptide derived from the human HIF-1a ODD that contains the target proline residue an exemplary such peptide has the sequence: Biotin-Acp-DLDLEALAP*YIPADDDFQL (SEQ ID NO:10). When this proline residue (P*) is hydroxylated, this peptide can be recognized as a substrate for [35S]-labeled human pVHL (M. Ivan et al., Science 292, 464 (2001); P. Jaakkola et al., Science 292, 468 (2001); F. Yu, S. B. White, F. S. Lee, Proc. Natl. Acad. Sci. U.S.A. 98, 9630 (2001)). Streptavidin-coated agarose beads can then be used to precipitate [35S]-labeled human pVHL associated with the biotinylated peptide and measured using scintillation counting.

In a preferred embodiment, the activity of the HPH proteins was measured using an assay dependent upon VBC-recognition of a hydroxylated peptide. The VBC protein complex was generated by co-expression of Glutathione S-Transferase-VHL (von Hippel Lindau), elongin B and elongin C in Escherichia coli followed by purification of the complex using glutathione affinity resin and size-exclusion chromatography. The HPH enzyme assay is conducted in the well of a 96- or 384-well plate. For the 384-well scale, the reaction (10 μl) contains buffer (20 mM MOPS (pH 6.5), 1.5 mM magnesium chloride, 5 mM potassium chloride, 0.1% bovine serum albumin, 1 mM TCEP, 10 μM ferrous ammonium sulfate, and 2 mM sodium ascorbate), substrate (2 μM α-ketoglutarate and 60 nM HIF-las peptide (biotin-DLDLEMLAPYIPMDDDFQL) (SEQ ID NO:11), enzyme (e.g. 8 nM of HPH3), and an inhibitor from a DMSO solution (1% DMSO final in the assay). After 45 min, the reaction is quenched by the addition of EDTA (2.5 mM) and the detection reagents (GST-VBC, Streptavidin-allophycocyanin and Europium-labeled anti-GST Antibody). The time-resolved fluorescent signal (excitation at 330 nm; emission at 620 and 665 nm) is read on a plate reader after 1 h of incubation. The ratio of signal at 665 nm to 620 nm is used to calculate the amount of peptide interaction with GST-VBC as a measure of protein hydroxylation.

These studies presented in the present application have identified the three human HPH enzymes from rhesus monkeys as attractive targets for use in the identification of unique therapeutic chemicals. It is known that that transgenic expression of a modified form of HIF-1α lacking the ODD in basal keratinocytes results in increased dermal vascularization (D. A. Elson et al., Genes Dev. 15, (2001)). The vascular bed formed under such conditions is stable and lacks the associated edema, inflammation and spontaneous hemorrhagic ulcers that accompany leaky vasculature resulting from the singular expression of vasculogenic growth factors such as VEGF (M. Detmar et al., J. Invest. Dermatol. 111, 1 (1998); F. Larcher, R. Murillas, M. Bolontrade, C. J. Conti, J. L. Jorcano, Oncogene 17 303, (1998); G. Thurston et al., Science 286, 2511 (1999)). The more substantive neovascularization resulting from constitutive HIF-1α expression may reflect the fact that this transcription factor activates not only VEGF gene expression but also other genes important for the formation of new blood vessels. Hence, selective inhibitors of the HPH enzymes of the invention provide useful leads for therapeutics capable of promoting angiogenesis.

### EXAMPLE

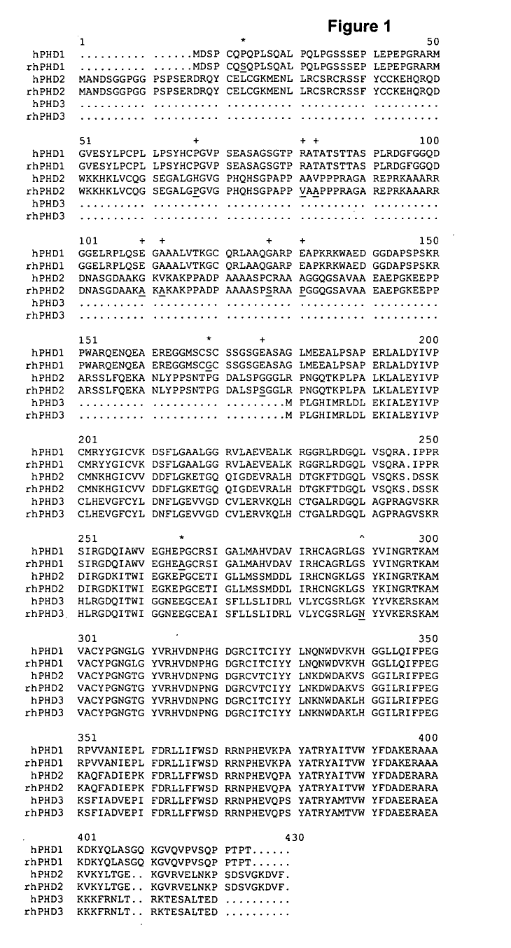

The Hypoxia inducible factor-Prolyl Hydroxylase (HPH) genes were cloned by using polymerase chain reaction mediated amplification of DNA from rhesus (Macaca mulatta) cDNA (rhesus gallbladder cDNA for HPH1, testis cDNA for HPH2 and HPH3) using primers designed on the basis of known HPH genes. The gene products were cloned into pETBlue-2 vectors and sequenced (FIG. 1). The genes are highly homologous to the human genes (98%, 99%, and 99.6% identical at the amino acid level to HPH1, 2 and 3, respectively).

For heterologous expression of these proteins, the genes were subcloned into pvl1393 vectors with either N- or C-terminal Glutathione S-Transferase (GST) tags to aid in purification of the proteins. SF9 cells were co-transfected with BaculoGold™ Linearized Baculovirus DNA (Becton Dickinson) and the complementing pvl1393 transfer vector containing the gene of interest. To identify recombinant virions, a plaque purification was completed and viral amplifications from one isolated plaque were done in SF9 cells. After two viral amplifications, sufficient amounts of a high titered pure virus stock were generated for large scale protein production. Expression verification using an anti-GST antibody was used at each step of the viral amplification process.

The use of N-terminal GST affinity tags increased our expression yields (˜10-fold in the case of HPH3) as compared to native proteins. For HPH3, it was discovered that the N-terminal GST tag reduced enzymatic activity (˜10-fold); in contrast to the HPH1 and HPH2 proteins, the GST-thrombin-HPH3 protein did not bind to a glutathione affinity column. The additional N-terminal extension of HPH1 and HPH2 as compared to HPH3 would suggest that a linker may be necessary to space the GST and HPH3 proteins to enable correct binding activity or structure of the GST protein. For HPH3, we used a C-terminal GST construct for expression as this protein could be bound to the glutathione affinity column for purification and the GST tag could be removed with thrombin proteolysis. Proteins were purified from the SF9 cell lysates by affinity chromatography on glutathione Sepharose 4B column, thrombin cleavage to release the protein from the GST tag, and then size-exclusion chromatography on a Superdex 75 column. The final yields of purified HPH protein were ˜1 mg/ml for HPH3 and ˜5 mg/ml for HPH1 and HPH2.

The activity of the HPH proteins was measured using an assay dependent upon VBC-recognition of a hydroxylated peptide. The VBC protein complex was generated by co-expression of Glutathione S-Transferase-VHL (von Hippel Lindau), elongin B and elongin C in Escherichia coli followed by purification of the complex using glutathione affinity resin and size-exclusion chromatography. The HPH enzyme assay is conducted in the well of a 96- or 384-well plate. For the 384 well scale, the reaction (10 μl) contains buffer (20 mM MOPS (pH 6.5), 1.5 mM magnesium chloride, 5 mM potassium chloride, 0.1% bovine serum albumin, 1 mM TCEP, 10 μM ferrous ammonium sulfate, and 2 mM sodium ascorbate), substrate (2 μM α-ketoglutarate and 60 nM HIF-1αs peptide (biotin-DLDLEMLAPYIPMDDDFQL), enzyme (e.g. 8 nM of HPH3), and an inhibitor from a DMSO solution (1% DMSO final in the assay). After 45 min, the reaction is quenched by the addition of EDTA (2.5 mM) and the detection reagents (GST-VBC, Streptavidin-allophycocyanin and Europium-labeled anti-GST Antibody). The time-resolved fluorescent signal (excitation at 330 nm; emission at 620 and 665 nm) is read on a plate reader after 1 h of incubation. The ratio of signal at 665 nm to 620 nm is used to calculate the amount of peptide interaction with GST-VBC as a measure of protein hydroxylation.

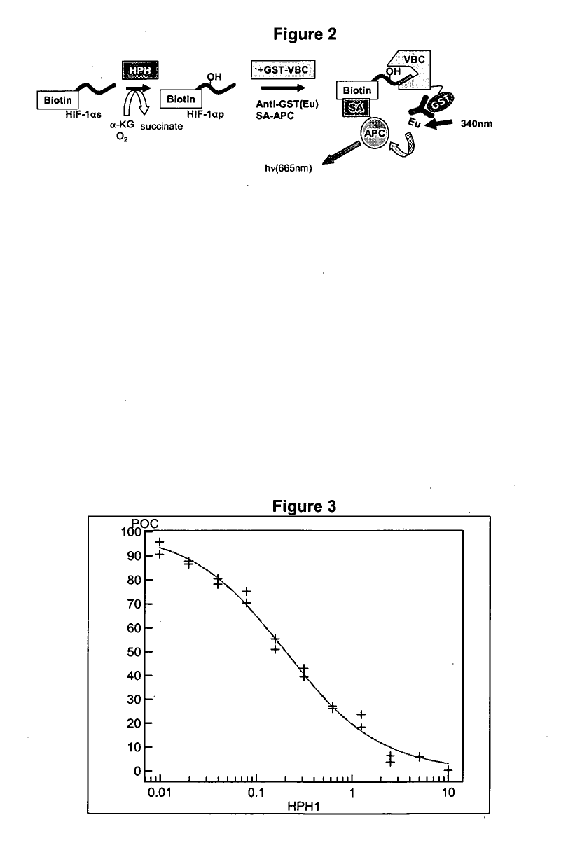

The assay schematic is shown in FIG. 2. The VBC protein is fused to the protein glutathione S-transferase (GST) and a europium-labeled anti-GST antibody is bound to GST while a streptavidin-allophycocyanin (SA-APC) conjugate is used to bind the biotinyl-peptide. The result of this complex assemble of proteins is that the fluorescent europium is brought close enough to the fluorescent APC molecule to permit Time-Resolved Fluorescent Resonant Energy Transfer (TR-FRET) at a unique wavelength (665 nm). The homogeneous nature of this assay provides for a faster and more reproducible assay than an ELISA format and one that is amenable to high-throughput screening.

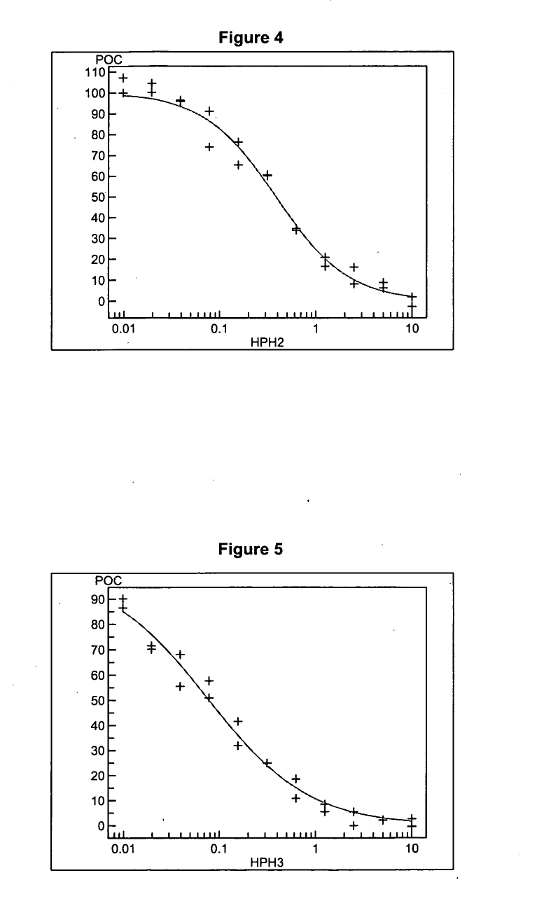

As exemplified by the inhibition of enzyme activity (FIGS. 3-5, Table 1), the recombinant rhesus HPH protein was catalytically active and exhibited a similar degree of inhibitor binding as the human counterparts. Similarly, we found that the addition of ascorbate and ferrous iron increased the activity of the enzyme whereas CoCl₂ inhibited enzyme activity. The specific activity of the recombinant enzymes was determined to be in the range of 3-10 nmol/min/mg which is in the range of previously reported activities from the human HPH enzymes (Tuckerman (2004) FEBS Lett. 576:145-150)

TABLE 1
Inhibitory activity of known HPH inhibitors against rhesus HPH.

| | IC₅₀ (μM) | | |
|---|---|---|---|
| | HPH1 | HPH2 | HPH3 |
| Succinate | 200 | 380 | 80 |
| Hydralazine | 400 | 320 | 54 |
| N-oxalylglycine | nd | nd | 0.66 |

1. An isolated nucleic acid sequence of SEQ ID NO: 1, SEQ ID NO:3 or SEQ ID NO:5.

2. An isolated nucleic acid sequence that encodes a protein of SEQ ID NO:2, SEQ ID NO:4 or SEQ ID NO:6.

3. An expression construct comprising a nucleic acid of claim 1 or claim 2 operably linked to a promoter.

4. An expression vector comprising a nucleic acid of claim 1 or claim 2.

5. A host cell transformed or transfected with an expression vector of claim 4 under conditions that allow expression of said nucleic acid in said host cell.

6. An isolated and purified protein comprising a sequence of SEQ ID NO:2, SEQ ID NO:4 or SEQ ID NO:6.

7. The protein of claim 6 wherein said protein is conjugated to a detectable label.

8. A method of producing a HIF prolyl hydroxylase protein comprising transforming a host cell with an expression vector of claim 5 under condition to allow expression of said nucleic acid sequence contained in said expression vector by said host cell; culturing said host cell to produce a protein product of said expressed nucleic acid sequence and isolating said protein product.

9. A method of increasing expression of a hypoxia inducible gene in a cell comprising contacting a cell that comprises a hypoxia inducible gene with an expression vector of claim 5 under conditions that allow expression of the nucleic acid sequence contained in the vector thereby providing for increased expression of a hypoxia inducible gene in said cell.

10. A method of assaying for hypoxia-inducible factor (HIF) prolyl hydroxylation, comprising the steps of:
a) incubating a mixture comprising an isolated HIF-specific prolyl hydroxylase (HPH) protein or recombinantly expressed HPH selected from the group consisting of a protein of SEQ ID NO:2, SEQ ID NO:4 and SEQ ID NO: 6, and a peptide substrate of HPH hydroxylase enzymes, under conditions whereby a proline residue on said substrate is hydroxylated, and b) detecting a resultant prolyl hydroxylation of the substrate.

11. The assay of claim 10, wherein the mixture further comprises a candidate agent which modulates the resultant prolyl hydroxylation.

12. The assay of claim 11, wherein the substrate comprises LAPY, wherein P is hydroxylated by the hydroxylase.

13. The assay of claim 12, wherein the hydroxylase is recombinantly expressed in a cell and the detecting step comprises detecting a transcriptional reporter of HIF dependent gene expression.

14. The assay of claim 12, wherein the hydroxylase is an isolated protein, and the detection step comprises detecting a reagent which selectively binds the prolyl hydroxylated substrate.

15. A method of screening for a modulator of HIF prolyl hydroxylation, comprising the steps of:
a) incubating a mixture comprising an isolated HIF-specific prolyl hydroxylase (HPH) protein or recombinantly expressed HPH selected from the group consisting of a protein of SEQ ID NO:2, SEQ ID NO:4 and SEQ ID NO: 6, and a peptide substrate of HPH hydroxylase enzymes, under conditions whereby a proline residue on said substrate is hydroxylated, and
b) detecting a resultant prolyl hydroxylation of the substrate;
wherein the incubating step is performed in the presence and absence of candidate modulator of prolyl hydroxylation wherein an increase in the prolyl hydroxylation in the presence of said candidate modulator is indicative of said modulator being an enhancer of prolyl hydroxylation and a decrease in the prolyl hydroxylation in the presence of said candidate modulator is indicative of said modulator being an inhibitor of prolyl hydroxylation.


SEQUENCE LISTING (13 sequences)

SEQ ID NO: 1 - DNA, 1281 bp, Macaca mulatta
  misc_feature: the nucleic acid sequence of rhesus HPH 1
atggccaatgacagcggcgggcccggcgggccgagcccgagcgagcgagaccggcagtactgcgagctgtgcgggaagatggagaacctgctgcgctgcagccgctgccgcagctccttctactgctgcaaggagcaccagcgtcaggactggaagaagcacaagctcgtgtgccagggcagcgagggcgccctcggccccggagtgggcccgcaccagcactccggccccgcgccgccggttgcagcgccgccgcccagggccggggcccgggagcccaggaaggcagcggcgcgccgggacaacgcctccggggacgcggccaaggcaaaagcaaaggccaagcccccggccgaccccgcggcggccgcgtcgccgtctcgcgcggccccgggcggccagggctcggcggtggctgccgaggccgagcccgggaaggaggagccgccggcccgctcatcgctgttccaggagaaggcgaacctgtaccccccgagcaacacgcccggggatgcgctgagccccagcggcggcctgcggcccaacgggcagacgaagcccctgccggcgctgaagctggcgctcgagtacatcgtgccgtgcatgaacaagcacggcatctgtgtggtggacgacttcctcggcaaggagaccggacagcagatcggcgacgaggtgcgcgccctgcacgacaccgggaagttcacggacgggcagctggtcagccagaagagcgactcgtccaaggacatccgaggcgataagatcacctggatcgagggcaaggagcccggctgcgaaaccattgggctgctcatgagcagcatggacgacctgatccgccactgtaacgggaagctgggcagctacaaaatcaatggccggacgaaagccatggttgcttgttatccgggcaatggaacgggttatgtacgtcatgttgataatccaaatggagatggaagatgtgtgacatgtatatattatcttaataaagactgggatgccaaggtaagtggaggtatacttcgaatttttccagaaggcaaagcccagtttgctgacattgaacccaaatttgatagactgctgtttttctggtctgaccgtcgcaaccctcatgaagtacaaccagcatatgctacaaggtacgcaataactgtttggtattttgatgcagatgagagagcacgagctaaagtaaaatatctaacaggtgaaaaaggtgtgagggttgaactcaataaaccttcagattcggtcggtaaagacgtcttctag

SEQ ID NO: 2 - protein, 407 aa, Macaca mulatta
  MISC_FEATURE: 2 rh HPH1 protein
MDSPCQSQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCGCSSGSGEASAGLMEEALPSAPERLALDYIVPCMRYYGICVKDSFLGAALGGRVLAEVEALKRGGRLRDGQLVSQRAIPPRSIRGDQIAWVEGHEAGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 3 - DNA, 1218 bp, Macaca mulatta
  misc_feature: he nucleic acid sequence of rhesus HPH 2
atggacagcccgtgccagtcgcagcccctaagtcaggctctccctcagttaccagggtcttcgtcagagcccttggagcctggccgggccaggatgggrgtggagagttacctgccctgtcccctgctcccctcctaccactgtccaggagtgcctagtgaggcctcagcggggagtgggactcccagagctacagccacctctaccactgccagcccccttcgggacggttttggcgggcaggatggtggtgagctgcggccgctgcagagtgaaggcgctgcagcgctggtcaccaaggggtgccagcgactggcagcccagggcgcccggcctgaggcccccaaacggaaatgggccgaggatggtggggatgccccttcacccagcaagcggccctgggccaggcaagagaaccaggaggcagagcgggagggtggcatgagctgcggctgcagcagtggcagtggtgaggccagtgctgggctgatggaggaggcgctgccctctgcacctgagcgcctggccctggactatatcgtgccctgcatgcggtactacggcatctgcgtcaaggacagcttcctgggggcagcgctgggcggccgcgtgctggccgaggtggaggccctcaaacggggtgggcgcctgcgagacgggcagctagtgagccagcgggcgatcccgccgcgcagcatccgtggggaccagattgcctgggtggaaggccatgaagcaggctgccgaagcattggtgccctcatggcccacgtggacgccgtcatccgccactgcgcagggcggctgggcagctatgtcatcaacgggcgcaccaaggccatggtggcgtgytacccaggcaacgggctcgggtacgtaaggcacgttgacaatccccacggcgatgggcgctgcatcacctgtatctattacctgaatcagaactgggacgttaaggtgcatggyggcctgctgcagatcttccctgagggccggccsgtggtagccaacatcgarccactctttgaccggttgctcattttctggtctgaccggcggaacccccacgaggtgaagccagcctatgccaccaggtacgccatcactgtctggtattttgatgccaaggagcgggcagcagccaaagacaagtatcagctagcatcaggacagaaaggtgtccaagtacctgtatcacagccgcctacgcccacctag

SEQ ID NO: 4 - protein, 426 aa, Macaca mulatta
  MISC_FEATURE: 4 rh PHD2
MANDSGGPGGPSPSERDRQYCELCGKMENLLRCSRCRSSFYCCKEHQRQDWKKHKLVCQGSEGALGPGVGPHQHSGPAPPVAAPPPRAGAREPRKAAARRDNASGDAAKAKAKAKPPADPAAAASPSRAAPGGQGSAVAAEAEPGKEEPPARSSLFQEKANLYPPSNTPGDALSPSGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 5 - DNA, 720 bp, Macaca mulatta
  misc_feature: nucleic acid sequence of rhesus HPH 3
atgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggagaccagatcacgtggatcgggggcaacgaggagggctgcgaggccatcagcttcctcttgtccctcatcgacaggctggtcctttactgcgggagccggctgggcaactactacgtcaaggagaggtctaaggcaatggtggcttgctatccgggaaatggaacaggttatgttcgccatgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgcaggaacccacacgaagtgcagccttcttacgcaaccagatatgctatgactgtctggtactttgatgctgaagaaagggcagaagccaaaaagaaattcaggaatttaactaggaaaactgaatctgccctcactgaagactga

SEQ ID NO: 6 - protein, 239 aa, Homo sapiens
  MISC_FEATURE: 6 rh PHD3
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGNYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 7 - protein, 407 aa, Homo Sapiens
MDSPCQPQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCSCSSGSGEASAGLMEEALPSAPERLALDYIVPCMRYYGICVKDSFLGAALGGRVLAEVEALKRGGRLRDGQLVSQRAIPPRSIRGDQIAWVEGHEPGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 8 - protein, 426 aa, Homo Sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 9 - protein, 239 aa, Homo Sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 10 - protein, 19 aa, Artificial Sequence
  peptide sequence
DLDLEALAPYIPADDDFQL

SEQ ID NO: 11 - protein, 19 aa, Artificial Sequence
  peptide sequence
DLDLEMLAPYIPMDDDFQL

SEQ ID NO: 12 - protein, 19 aa, Artificial Sequence
  peptide sequence
DLDLEMLAPYIGMDDDFQL

SEQ ID NO: 13 - protein, 19 aa, Artificial Sequence
  peptide sequence
DLDLEALAPYIGADDDFQL